(19) United States
(12) Patent Application Publication
(10) Pub. No.: US 2004/0013695 A1
(43) Pub. Date: Jan. 22, 2004

(54) Oral solid dose vaccine

(76) Inventor: Vincent Vande-Velde, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/344,798

(22) PCT Filed: Aug. 14, 2001
(86) PCT No.: PCT/IB01/01711

(30) Foreign Application Priority Data
Aug. 15, 2000 (GB) 0020089.91

(51) Int. Cl. A61K 33/00; A61K 9/00

(52) U.S. Cl. 424/400; 424/600

(57) ABSTRACT

The present invention relates to novel vaccine formulations suitable for oral administration. The vaccine formulations are in a solid form comprising antigen and suitable excipients, which after insertion into the mouth, rapidly dissolve in saliva, thereby releasing the vaccine into the mouth. Specifically, the solid form may consist of a cake of vaccine which is formed from a liquid solution or suspension by sublimation, preferably sublimation by lyophilisation. Preferred vaccines are those containing antigens which are or are derived from pathogens that normally infect or invade the host through a mucosal membrane, or those vaccines that further comprise an antacid. Particularly preferred vaccines are combination vaccines that comprise more than one antigen, and more preferably when the antigens are from more than one pathogen.

The present invention relates to novel vaccine formulations suitable for oral administration. The vaccine formulations are in a solid form comprising antigen and suitable excipients, which after insertion into the mouth, rapidly dissolve in saliva, thereby releasing the vaccine into the mouth. Specifically, the solid form may consist of a cake of vaccine which is formed from a liquid solution or suspension by sublimation, preferably sublimation by lyophilisation. Preferred vaccines are those containing antigens which are or are derived from pathogens that normally infect or invade the host through a mucosal membrane, or those vaccines that further comprise an antacid. Particularly preferred vaccines are combination vaccines that comprise more than one antigen, and more preferably when the antigens are from more than one pathogen.

Mucosal vaccination has received a great deal of attention from researchers over recent years, and amongst the most investigated areas of mucosal vaccination has been the selection of the route of administration. For example, vaccines have commonly been administered through the nasal or oral routes (Mestecky, J. 1987, Journal of Clinical Immunology, 7, 265-276). For oral vaccination, one major consideration is how to avoid antigenic degeneration by stomach acid. Accordingly, oral vaccines commonly are liquid vaccine formulations in large volumes containing an antacid to neutralise stomach acids, or alternatively they contain vehicles, such as microspheres, that protect the antigen by encapsulation. Liquid live attenuated virus vaccines have been administered orally for many years, examples of which include polio virus vaccine which is administered to infants in a drop form.

In all of these forms of vaccination, the administration of a liquid into the mouth is associated with problems. For example, administering liquid into the mouths of babies is often problematical, especially when the taste of the vaccine is unpleasant. Likewise, administration of tablets or gelatin capsules containing the vaccine to babies or adults is often difficult. In all of these forms of administration there is a possibility that the vaccine is spat out or that the tablet is not able to be swallowed. Accordingly, there is a need to develop an alternative form of oral vaccine delivery.

The present invention resides in the finding that oral vaccination is possible with solid vaccine formulations which dissolve rapidly in saliva after insertion into the mouth. Preferably the time period before complete dissolution is such that the solid formulation may not be swallowed or spat out before the vaccine is dispersed into the saliva.

The solid vaccine forms of the present invention are porous solid forms, termed “cakes”, which are small enough to be placed in the mouth, or under the tongue. The vaccine cakes of the present invention are formed from a liquid solution or suspension of vaccine by sublimation, and in a preferred form of the invention the sublimation is performed by lyophilisation. This flash dissolution preferably takes place before the vaccinee is able to reject the cake by spitting it out, or able to swallow the undissolved cake. Preferably the time of dissolution of the cake is less than 10 seconds, more preferably less than 5 seconds, and preferably less than 2 seconds and most preferably in less than 1 second.

In another aspect of the present invention the oral vaccine quick dissolving cake comprises an antacid. The antacid being such that when dissolved in saliva, and swallowed, it is capable of raising the pH of the stomach contents such that the vaccine antigen is not substantially degraded in the stomach. Most preferably the antacid is water insoluble and also acts as an adjuvant, in addition it is more preferred that when antigen is adsorbed to the surface of the insoluble antacid/adjuvant the antigen is protected from stomach acid.

GB1548022A and GB 2111423B describe solid pharmaceutical dosage forms being in the form of a quick dissolving pill. U.S. Pat. No. 5,039,540; U.S. Pat. No. 4,946,684; U.S. Pat. No. 5,976,577 and WO 99/02140 describe rapidly dissolving pharmaceutical dosage forms prepared by lyophilisation. Seager also describes one such dosage form in J. Pharm. Pharmacol., 1998, 50: 375-382.

WO 00/00218 describes the mouth as being a route of administration for vaccines which are intended to generate strong local immune responses in the mouth and also other mucosal tissues. These formulations preferably contain an absorbent excipient that holds the vaccine within the mouth, or abrades the buccal mucosa, both designed to enhance the uptake of antigen across the buccal mucous membrane.

The quick dissolving vaccine cakes of the present invention are formed by sublimation of a liquid vaccine formulation. Generally, this process is performed by lyophilisation, although ambient temperature sublimation is encompassed within the present invention. As such the vaccine cakes of the present invention are manufactured by formulating the vaccine in a liquid form, followed by aliquoting the liquid into discrete dosage forms, followed by sublimation to remove the liquid. The removal of the liquid does not substantially reduce the volume of the dosage form, and as such leaves an extremely porous cake that exposes a large surface area to saliva in the mouth. The antigen encapsulated therein, is able to be swallowed after dissolution in saliva such that it may be sampled by the oral or pharangeal, or intestinal mucosal immune tissues, thereby stimulating an immune response.

The formulations of the vaccine cakes may be any of those described below, but may also encompass those described in GB 1548022A; GB 2111423B; U.S. Pat. No. 5,039,540; U.S. Pat. No. 4,946,684; U.S. Pat. No. 5,976,577; WO 99/02140; or Seager, J. Pharm. Pharmacol., 1998, 50: 375-382. The cakes are preferably lyophilised, and may be made by the technique of forming viscous solutions of vaccine which are then separated into discrete dosage forms (followed by conventional lyophilisation); or more preferably the liquid vaccine formulation may be poured into individual wells followed by sublimation by lyophilisation. After lyophilisation, the water is removed to leave the rapidly dissolvable vaccine cakes in the well which then can either be removed, or sealed within the well to form a blister pack.

The technique of lyophilisation, and details of other suitable excipients, may be found in Cameron et al., “Good Pharmaceutical freeze-drying Practice”, Interpharm, Buffalo Grove (1997).

It is foreseen that compositions of the present invention will be used to formulate vaccines containing antigens derived from a wide variety of sources. For example, antigens may include human, bacterial, or viral nucleic acid, pathogen derived antigen or antigenic preparations, tumour derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen, which antigen or antigenic composition is derived from HIV-1, (such as tat, nef, gp120 or gp160), human herpes viruses, such as gD or derivatives thereof or Immediate Early protein such as ICP27 from HSV1 or HSV2, cytomegalovirus ((esp Human) (such as gB or derivatives thereof), Epstein Barr virus (such as gp350 or derivatives thereof), Varicella Zoster Virus (such as gpI, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, . . . ), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or Vero cells or whole flu virosomes (as described by R. Gluck, Vaccine, 1992, 10, 915-920) or purified or recombinant proteins thereof, such as HA, NP, NA, or M proteins, or combinations thereof), or derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans. In a preferred aspect of the invention, the rapidly dissolving vaccine cake for oral administration does not comprise rotavirus.

Preferred bacterial vaccines comprise antigens derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof. Other preferred bacterial vaccines comprise antigens derived from Morexella Catarrhalis (including outer membrane vesicles thereof, and OMP106 (WO97/41731)) and from Neisseria mengitidis B (including outer membrane vesicles thereof, and NspA (WO 96/29412).

Particularly preferred vaccines are combination vaccines that comprise more than one antigen, and more preferably when the antigens are from more than one pathogen. By way of example, a lyophilised measles, mumps and rubella vaccine may be produced, suitably in a formulation comprising 8% sucrose, 2% manitol and 1.4% amino acid mix.

Derivatives of Hepatitis B Surface antigen are well known in the art and include, inter alia, those PreS1, PreS2 S antigens set forth described in European Patent applications EP-A-414 374; EP-A-0304 578, and EP 198-474. In one preferred aspect the vaccine formulation of the invention comprises the HIV-1 antigen, gp120, especially when expressed in CHO cells. In a further embodiment, the vaccine formulation of the invention comprises gD2t as hereinabove defined.

In a preferred embodiment of the present invention vaccines containing the claimed adjuvant comprise antigen derived from the Human Papilloma Virus (HPV) considered to be responsible for genital warts, (HPV 6 or HPV 11 and others), and the HPV viruses responsible for cervical cancer (HPV16, HPV18 and others).

Particularly preferred forms of genital wart prophylactic, or therapeutic, vaccine comprise L1 particles or capsomers, and fusion proteins comprising one or more antigens selected from the HPV 6 and HPV 11 proteins E6, E7, L1, and L2.

The most preferred forms of fusion protein are: L2E7 as disclosed in WO 96/26277, and protein D (⅓)-E7 disclosed in GB 9717953.5 (PCT/EP98/05285).

A preferred HPV cervical infection or cancer, prophylaxis or therapeutic vaccine, composition may comprise HPV 16 or 18 antigens. For example, L1 or L2 antigen monomers, or L1 or L2 antigens presented together as a virus like particle (VLP) or the L1 alone protein presented alone in a VLP or capsomer structure. Such antigens, virus like particles and capsomer are per se known. See for example WO94/00152, WO94/20137, WO94/05792, and WO93/02184.

Preferred is HPV 16 and/or 18 lyophilised in a the presence of a sugar such as sucrose, suitably at 31.5%, maltose suitably at 3.15%, trehalose suitably at 3.15% and most preferably a mix of sucrose and maltitol, suitably with sucrose at 3.15% and maltitol at 0.8%.

Additional early proteins may be included alone or as fusion proteins such as preferably E7, E2 or E5 for example; particularly preferred embodiments of this includes a VLP comprising L1E7 fusion proteins (WO 96/11272).

Particularly preferred HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D-E6/E7 fusion. Such vaccine may optionally contain either or both E6 and E7 proteins from HPV 18, preferably in the form of a Protein D-E6 or Protein D-E7 fusion protein or Protein D E6/E7 fusion protein.

The vaccine of the present invention may additionally comprise antigens from other HPV strains, preferably from strains HPV 6, 11, 31, 33, or 45.

Vaccines of the present invention further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. It's full structure is disclosed in the International Patent Application No. PC/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. A preferred embodiment of the present invention is a Malaria vaccine wherein the antigenic preparation comprises a combination of the RTS,S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The formulations may also contain an anti-tumour antigen and be useful for the immunotherapeutic treatment cancers. For example, the adjuvant formulation finds utility with tumour rejection antigens such as those for prostrate, breast, colorectal, lung, pancreatic, renal or melanoma cancers. Exemplary antigens include MAGE 1 and MAGE 3 or other MAGE antigens for the treatment of melanoma, PRAME, BAGE or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma. Other Tumor-Specific antigens are suitable for use with adjuvant of the present invention and include, but are not restricted to Prostate specific antigen (PSA) or Her-2/neu, KSA (GA733), MUC-1 and carcinoembryonic antigen (CEA). Accordingly in one aspect of the present invention there is provided a vaccine comprising an adjuvant composition according to the invention and a tumour rejection antigen.

Additionally said antigen may be a self peptide hormone such as whole length Gonadotrophin hormone releasing hormone (GnRH, WO 95/20600), a short 10 amino acid long peptide, in the treatment of many cancers, or in immunocastration.

It is foreseen that compositions of the present invention will be used to formulate vaccines containing antigens derived from Borrelia sp.. For example, antigens may include nucleic acid, pathogen derived antigen or antigenic preparations, recombinantly produced protein or peptides, and chimeric fusion proteins. In particular the antigen is OspA. The OspA may be a full mature protein in a lipidated form virtue of the host cell (E. coli) termed (Lipo-OspA) or a non-lipidated derivative. Such non-lipidated derivatives include the non-lipidated NS1-OspA fusion protein which has the first 81 N-terminal amino acids of the non-structural protein (NS1) of the influenza virus, and the complete OspA protein, and another, MDP-OspA is a non-lipidated form of OspA carrying 3 additional N-terminal amino acids.

Vaccines of the present invention may be used for the prophylaxis or therapy of allergy. Such vaccines would comprise allergen specific (for example Der p1) and allergen non-specific antigens (for example peptides derived from human IgE, including but not restricted to the stanworth decapeptide (EP 0 477 231 B1)).

In particular, the preferred antigens are those which are, or are derived from, pathogens that infect a mucosal surface. In particular, polio, RSV, Campylobacter, ETEC, Helicobacter, Chlamidia, and influenza are preferred antigens.

In some embodiments of the present invention, the antigens will be formulated with a pharmaceutical carrier. Suitable pharmaceutical carriers for use in the vaccine according to the invention include those known in the art as being suitable for oral administration, especially to infants. Such carriers include and are not limited to carbohydrates, polyalcohols, amino acids, aluminium hydroxide or phosphate, magnesium hydroxide or phosphate, hydroxyapatite, talc, titanium oxide, iron hydroxide or phosphate, magnesium stearate, carboxymethylcellulose, hydroxypropylmethylcellulose, microcrystalline cellulose, gelatin, vegetal peptone, xanthane, caraghenane, arabic gum, β-cyclodextrin.

When it is desired that the antigen should reach mucosal tissues beyond the stomach, it is a preferred aspect of the present invention that the vaccine cake should contain an antacid. Suitable for use as antacids in the vaccine of the invention are organic antacids such as organic acid carboxylate salts. A preferred antacid in the vaccine composition of the invention contains an organic acid carboxylate salt, preferably a salt of citric acid such as sodium citrate or potassium citrate. Another suitable antacid is aluminium hydroxide or phosphate. Other, suitable antacid components include inorganic antacids for example aluminium hydroxide Al(OH)₃ and magnesium hydroxide Mg(OH)₂. Commercially available antacids which are suitable for use in the invention include Mylanta (trade mark) which contains aluminium hydroxide and magnesium hydroxide. These are insoluble in water and are given in suspension.

A particularly preferred antacid that may be used in the vaccine composition of the present invention is the insoluble inorganic salt, calcium carbonate (CaCO₃). The calcium carbonate is able to associate with the antigen and the antigenic activity is maintained during the association with the calcium carbonate.

It may also be advantageous to formulate the virus of the invention in lipid-based vehicles such as virosomes or liposomes, in oil in water emulsions or with carrier particles. Alternatively or in addition immunostimulants such as those known in the art for oral vaccines may be included in the formulation. Such immunostimulants include bacterial toxins, particularly cholera toxin (CT) in the form of the holotoxin (entire molecule) or the B chain only (CTB) and the heat labile enterotoxin of E. coli (LT). Mutated LTs (mLTs) which are less likely to convert to their active form than the native LT are described in WO 96/06627, WO 93/13202 and U.S. Pat. No. 5,182,109.

Further immunostimulants which may advantageously be included are saponin derivatives such as QS21 and monophosphoryl lipid A, in particular 3-de-O-acylated monophosphoryl lipid A (3D-MPL). Purified saponins as oral adjuvants are described in WO 98/56415. Saponins and monophosphoryl lipid A may be employed separately or in combination (e.g. WO 94/00153) and may be formulated in adjuvant systems together with other agents. 3D-MPL is a well-known adjuvant manufactured by Ribi Immunochem, Montana and its manufacture is described in GB 2122204.

Aluminium hydroxide is a particularly preferred component of a vaccine composition according to the invention as it can provide not only an antacid effect but also an adjuvantation effect.

To prevent sedimentation of calcium carbonate during the filling step, viscous agents are preferably present in the formulation. Possible viscous agents that may be used include pseudoplastic excipients. A pseudoplastic solution is defined as a solution having higher viscosity on standing compared to its viscosity under agitation. Excipients of this type are natural polymers such as arabic gum, adragante gum, agar-agar, alginates, pectines or semi-synthetic polymers for example: carboxymethylcellulose (Tyloses C®), methylcellulose (Methocels A®, Viscotrans MC®, Tylose MH® and MB®), hydroxypropylcellulose (Klucels®), and hydroxypropylmethylcellulose (Methocels E® and K®, Viscontrans MPHC®). In general those pseudoplastic excipients are used together with thixotropic agents. Alternative viscous agents that may be used are pseudoplastic excipients with low flowing capacity. Those polymers, at a sufficient concentration, give rise to a structural fluid arrangement resulting in a high viscosity solution having low flowing capacity on standing. A certain quantity of energy needs to be given to the system to allow flowing and transfer. External energies (agitation) are needed to destroy temporarily the structural fluid arrangement in order to obtain a fluid solution.

Examples of such polymers are Carbopols® and xanthane gum. Thixotropic excipents, may also be used, which become a gel structure on standing whilst under agitation they form a fluid solution. Examples of thixotropic excipients are: Veegum®(Magnesium-aluminium silicate) and Avicel RC® (about 89% microcrystalline cellulose and 11% Carboxymethylcellulose Na).

In order to enhance the physical stability of the cake structure binding agents may be used such as dextran. Increasing molecular weight of the dextran, increases the integrity of the vaccine cake. As such, Dextran 10 is a polymer having an average molecular weight around 10 000 and is suitable for use in the present invention, also dextrans having a molecular weight of 70 000; 100 000; and 400 000 may be used. β-cyclodextrine may also be used as a binding agent.

The vaccine composition of the present invention preferably comprises a viscous agent selected from xanthane gum or starch.

Thus the vaccine composition of the present invention is preferably formulated with a combination of calcium carbonate and xanthane gum, both with and without dextran binding agent. Also preferred are vaccine formulations comprising dextran and xanthane gum and/or dextran and calcium carbonate or aluminium salts such as aluminium hydroxide.

Other components of a composition used in the invention suitably include glass forming compounds to stabilise the vaccine formulation during storage. Examples of such compounds such as glass forming polyols such as those described in U.S. Pat. No. 5,098,893, U.S. Pat. No. 6,071,428; WO 98/16205; WO 96/05809; WO 96/03978; U.S. Pat. No. 4,891,319; U.S. Pat. No. 5,621,094; WO 96/33744. In particular, sugars, including mono, di, tri, or oligo saccharides and their corresponding sugar alcohols are preferred. Suitable sugars for use in the present invention are well known in the art and include, trehalose, sucrose, lactose, fructose, galactose, mannose, maltulose, iso-maltulose and lactulose, maltose, or dextrose and sugar alcohols of the aforementioned such as mannitol, lactitol and maltitol.

The vaccine composition according to the invention may contain additional components including for example flavourings (particularly for an oral vaccine) and bacteriostatic agents.

Lyophilised formulations may conveniently be provided in the form of tablets in a pharmaceutical blister pack.

In another aspect the invention provides a composition comprising a live attenuated bacterium or virus, or live viral or bacterial vector, wherein the composition is a lyophilised solid capable of immediate dissolution when placed in the mouth.

Vaccines of the invention may be formulated and administered by known techniques, using a suitable amount of live virus to provide effective protection against infection without significant adverse side effects in typical vaccinees. A suitable amount of live virus will normally be between 10⁴ and 10⁷ ffu per dose. A typical dose of vaccine may comprise 10⁵-10⁶ ffu per dose and may be given in several doses over a period of time, for example in two doses given with a two-month interval. Benefits may however be obtained by having more than 2 doses, for example a 3 or 4 dose regimen, particularly in developing countries. The interval between doses may be more or less than two months long. An optimal amount of live virus for a single dose or for a multiple dose regimen, and optimal timing for the doses, can be ascertained by standard studies involving observation of antibody titres and other responses in subjects.

The amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, most preferably 1 to 50 μg. An optimal amount for a particular vaccine can be ascertained by standard studies involving observation of appropriate immune responses in subjects. Following an initial vaccination, subjects may receive one or several booster immunisation adequately spaced.

The oral solid dose forms of the present invention have a relatively low volume to ease insertion into the mouth or under the tongue. As such the liquid vaccine is aliquoted in volumes of about 0.1 to 1 ml, preferably 0.1 to 0.5 ml, and most preferably in the range of 0.1 to 0.3 ml.

The present invention is illustrated by the following examples.

### EXAMPLE 1

Lyophilised Virus with Al(OH)₃ or CaCO₃ for Blister Presentation

A reference known virus was used throughout these examples, standard techniques are used for preparing virus doses. Frozen purified viral bulk is thawed and diluted with appropriate medium composition, in this case Dulbecco's modified eagle Medium, up to a desired standard viral concentration, in this case 10^{6.2} ffu/ml. Aluminium hydroxide or Calcium carbonate suspension is added to reach a final quantity of 48 mg/dose and the virus composition is diluted with lyophilisation stabiliser which may be sucrose, dextran or amino-acid 4%, or gelatin, or vegetal peptone, or xanthane up to the target viral titre of 10^{5.6} ffu/dose. An aseptic filling operation is employed to transfer doses of 0.5 ml or preferably less to plastic blister cavities. The composition is lyophilised, and the blister cavities are sealed by thermic sealing.

Optionally standard ingredients are included to prevent the aluminium hydroxide suspension from settling. Such standard ingredients include for example magnesium stearate, carboxymethylcellulose, hydroxypropylmethylcellulose, microcrystalline cellulose, and silicone polymers. Flavourings may also be included.

The following formulations were made, and tested for virus titre before and after lyophilisation into a “cake” and storage for 1 week at 37° C. These formulations dissolve rapidly in the mouth.

| | | Viral | Viral titer after |
|---|---|---|---|
| | | titer before | lyophilisation and |
| Batch n° | Fomulation composition | lyophilisation | 1 week at 37° |
| 99B10/06 | Sucrose 4% | 10^{5.11} | 10^{4.53} |
| | Sodium glutamate 3.7% | | |
| | Al(OH)3 48 mg | | |
| 99C11/12 | Maltitol 3% | 10^{4.16} | 10^{3.79} |
| | Al(OH) 48 mg | | |
| | Hydroxypropylmethyl- | | |
| | cellulose: 1% | | |
| 00C24/05 | Sucrose: 2% | 10^{5.02} | 10^{4.54} |
| | Dextran: 4% | | |
| | Sorbitol: 3% | | |
| | Am. Acids: 2% | | |
| | CaCO₃: 60 mg | | |
| | Xanthane 0.3% | | |
| 00C24/06 | Sucrose: 2% | 10^{4.86} | 10^{4.56} |
| | Dextran: 4% | | |
| | Sorbitol: 3% | | |
| | Am. Acids: 2% | | |
| | CaCO₃: 60 mg | | |
| | Xanthane 0.3% | | |
| 00F26/11 | Sucrose: 1% | 10^{4.70} | 10^{4.40} |
| | Dextran: 2% | | |
| | Sorbitol: 1.5% | | |
| | Am. Acids: 1% | | |
| | CaCO₃: 60 mg | | |
| | Starch: 2% | | |

### EXAMPLE 2

Lyophilised Virus with Antacid for Blister Presentation

The vaccine cake formulations were prepared in 0.6 ml volumes as described as in example 1, whilst the lyophilisation cycle was performed as follows.

[image]

The formulations were tested for physical stability and speed of dissolution in the mouth.

| | | | | | Amino | | | | |
|---|---|---|---|---|---|---|---|---|---|
| | Mylanta | lactose | dextran | sorbitol | Acid | Histidine | Al(OH)₃ | Cake | Speed of |
| | 64 mg | 10 mg | 20 mg | 15 mg | 10 mg | 72 mg | 42 mg | aspect | disolution |
| 01 | + | − | − | − | − | − | − | OK, but | medium |
| | | | | | | | | soft | |
| 02 | + | + | + | + | + | + | + | hard | slow |
| 03 | − | + | + | + | + | + | + | hard | medium |
| 04 | − | − | + | + | + | + | + | hard | medium |
| | | | 12.6% | 9.4% | 6.3% | 45.3% | 26.4% | | |
| 05 | − | + | − | + | + | + | + | fragile | quick |
| | | | | 10.8% | 7.2% | 51.8 | 30.2 | | |
| 06 | − | + | + | − | + | + | + | fragile | medium |
| | | | 13.9 | | 6.9% | 50% | 29.1% | | |
| 07 | − | + | + | + | − | + | + | fragile | slow |
| 08 | − | − | − | + | + | + | + | fragile | quick |
| 09 | − | + | − | − | + | + | + | fragile | quick |
| 10 | − | + | + | − | − | + | + | fragile | medium |
| 11 | − | − | + | + | − | + | + | fragile | quick |
| 12 | − | − | + | − | + | + | + | − | − |
| 13 | − | + | − | + | − | + | + | fragile | quick |
| 14 | − | − | − | − | + | + | + | fragile | quick |
| 15 | − | + | − | − | − | + | + | fragile | quick |
| 16 | − | − | + | − | − | + | + | fragile, | quick |
| 17 | − | − | − | + | − | + | + | fragile | quick |
| 18 | − | − | − | − | − | + | + | fragile | quick |

Speed of dissolution: Quick = the cake dissolve so quickly that there is no time to swallow it, or to spit it out; Medium = there is time to swallow or to spilt out part(s) of the lyophilised cake; Slow = there is time to swallow or to spilt out most of the lyophilised cake. Fragile = means that it will be difficult to take the cake out of the blister in one piece.

### EXAMPLE 3

Dextran Containing Cakes

Vaccine cakes were prepared without or without dextran as binding agent, and tested for cake aspect and stabilisation of virus titre.

| | | | | Viral | | |
|---|---|---|---|---|---|---|
| | | Target | | titer | Viral | Viral titer |
| | | viral | Cake | liquid | titer | 1 week |
| Batch n° | Composition | titer | aspect | formul. | lyophi. | 37° C. |
| 99B10/06 | S Glu Al(OH)3 | 5.43 | friable | 5.11 | | 4.53 |
| 99B10/08 | S Glu PO₄ | 5.43 | friable | | | |
| | Al(OH)₃ | | | | | |
| 99C11/12 | M Al(OH)₃ | 5.58 | friable | 4.6 | <3.44 | 3.79 |
| | HPMC 1% | | | | | |
| 99C11/13 | M Al(OH)₃ | 5.58 | friable | | | |
| | HPMC 0.2% | | | | | |
| 99C17/10 | S D Al(OH)₃ | 5.6 | good + | | | |
| | HPMC 1% | | powder | | | |
| 99C17/11 | S D Al(OH)₃ | 5.6 | good + | | | |
| | | | powder | | | |
| 99D29/16 | D Ppea Al(OH)₃ | 5.59 | | | | |
| 99D29/17 | D Xanth. Al(OH)₃ | 5.59 | | | | |

S = sucrose; Glu = Na glutamate; M = maltitol; D = Dextran; Ppea = Pea peptone; Xanth = xanthane; HPMC = hydroxipropylmethylcellulose.

Although the cakes not containing dextran were solid and suitable for vaccine formulations, the addition of dextran hardened the cake such that they were suitable for use in a blister pack.

### EXAMPLE 4

Lyophilisation of Virus in Presence of CaCO₃ Antacid

| | | Viral titer at time = | Viral titer after |
|---|---|---|---|
| | | zero after | lyophilisation and 1 |
| Batch n° | Composition | lyophilisation | week at 37° C. |
| 99K08/01 | Sucrose: 2% | 10^{5.28} | 10^{5.10} |
| | Dextran: 4% | | |
| | Sorbitol: 3% | | |
| | Am. Acids: 2% | | |
| | CaCO₃: 50 mg | | |
| 99K08/02 | Sucrose: 2% | 10^{5.16} | 10^{5.15} |
| | Dextran: 4% | | |
| | Sorbitol: 3% | | |
| | Am. Acids: 2% | | |
| | CaCO₃: 60 mg | | |
| 00C24/01 | Sucrose: 2% | 10^{5.07} | 10^{4.69} |
| | Dextran: 4% | | |
| | Sorbitol: 3% | | |
| | Am. Acids: 2% | | |
| | CaCO₃: 60 mg | | |
| | Xanthane 0.3% | | |
| 00C24/03 | Sucrose: 2% | 10^{5.07} | 10^{4.85} |
| | Dextran: 4% | | |
| | Sorbitol: 3% | | |
| | Am. Acids: 2% | | |
| | CaCO₃: 60 mg | | |
| | Xanthane 0.3% | | |
| 00E09/25 | Sucrose: 2% | 10^{5.03} | 10^{4.91} |
| | Dextran: 4% | | |
| | Sorbitol: 3% | | |
| | Am. Acids: 2% | | |
| | CaCO₃: 60 mg | | |
| | Xanthane 0.25% | | |
| 00E09/30 | Sucrose: 2% | 10^{5.01} | 10^{4.87} |
| | Dextran: 4% | | |
| | Sorbitol: 3% | | |
| | Am. Acids: 2% | | |
| | CaCO₃: 60 mg | | |
| | Xanthane 0.30% | | |
| 00F26/06 | Sucrose: 2% | 10^{4.50} | 10^{4.70} |
| | Dextran: 4% | | |
| | Sorbitol: 3% | | |
| | Am. Acids: 2% | | |
| | CaCO₃: 60 mg | | |
| | Starch: 2% | | |

This is the “all in one”: lyophilisation of virus and antacid (CaCO3) together in the same vial. Rotavirus activity is maintained even in presence of Xanthane gum and Starch (both used to prevent sedimentation during the filling step).

### EXAMPLE 5

Lyophilised Tablets for Quick Disintegration when Placed in the Mouth

Other suitable formulations were tested using the techniques described above, and were found to be suitable for the vaccines of the present invention.

| | | | Viral titer after |
|---|---|---|---|
| | Fomulation | Viral titer before | lyophilisation and |
| Batch n° | composition | lyophilisation | 1 week at 37° |
| 99B10/06 | Sucrose 4% | 10^{5.11} | 10^{4.53} |
| | Sodium glutamate | | |
| | 3.7% | | |
| | Al(OH)3 48 mg | | |
| 99C11/12 | Maltitol 3% | 10^{4.16} | 10^{3.79} |
| | Al(OH) 48 mg | | |
| | Hydroxypropyl- | | |
| | methyl- | | |
| | cellulose: 1% | | |
| | | Viral titer at time = | Viral titer after |
| | Fomulation | zero after | lyophilisation and |
| Batch n° | composition | lyophilisation | 1 week at 37° |
| 00C24/05 | Sucrose: 2% | 10^{5.02} | 10^{4.54} |
| | Dextran: 4% | | |
| | Sorbitol: 3% | | |
| | Am. Acids: 2% | | |
| | CaCO₃: 60 mg | | |
| | Xanthane 0.3% | | |
| 00C24/06 | Sucrose: 2% | 10^{4.86} | 10^{4.56} |
| | Dextran: 4% | | |
| | Sorbitol: 3% | | |
| | Am. Acids: 2% | | |
| | CaCO₃: 60 mg | | |
| | Xanthane 0.3% | | |
| 00F26/11 | Sucrose: 1% | 10^{4.70} | 10^{4.40} |
| | Dextran: 2% | | |
| | Sorbitol: 1.5% | | |
| | Am. Acids: 1% | | |
| | CaCO₃: 60 mg | | |
| | Starch: 2% | | |

In the “lyoc concept” both Xanthane and Starch can be used (maintaining the quick dissolution properties of the lyophilised cake).

### EXAMPLE 6

Oral Vaccination of Mice with OspA Lyoc

The following lyophilised fast dissolving tablets were prepared:

| Group | Description |
|---|---|
| 1 | SDSA, CaCO3 8 mg, Xanthane 0.3%, Lipo-OspA 10 μg |
| 2 | SDSA, CaCO3 8 mg, Xanthane 0.3%, Lipo-OspA 10 μg, |
| | LT 2.5 μg |
| 3 | SDSA, CaCO3 8 mg, Xanthane 0.3%, Lipo-OspA 10 μg, |
| | Laureth-9 0.5% |
| 4 | SDSA, CaCO3 8 mg, Xanthane 0.3%, Lipo-OspA 10 μg, |
| | MPL 5 μg |
| 5 | SDSA, CaCO3 8 mg, Xanthane 0.3%, Lipo-OspA 10 μg, |
| | Laureth-9 0.5%, MPL 5 μg |

SDSA = mixture of Sucrose 2%
Dextran-40000 4%
Sorbitol 3%
Amino acids 2%

Experimental Procedure

Eight week old Balb/c mice were primed at day 0 by an intramuscular (IM) administration of 1 μg Lipo-OspA adsorbed onto 50 μg aluminium hydroxyde. Groups of 8 mice were boosted at day 28 either orally with the lyoc formulations described above or intramuscularly with 1 μg Lipo-OspA adsorbed onto 50 μg aluminium hydroxyde (positive control). A second boost was done with lyoc formulations at day 56. Serum IgG antibodies as well fecal IgA were measured by ELISA.

Results

In general the oral lyoc formulations elicited lower serum IgG responses than the OspA IM booster. However, all lyoc formulations induced a significant immune response after each boosting, the magnitude of the observed peak immune responses after each subsequent boosting dose was greater than the peak observed after the previous boosting dose. All Groups 1 to 5 had approximately 20-25 μg/ml of OspA specific IgG in their serum after the second boost.

### EXAMPLE 7

Oral Vaccination of Mice with Influenza Antigens

5 different samples were prepared.

All samples contain 30 μg HA of A/Beijing/262/95 whole virus

Sucrose 2%

Sorbitol 3%

Dextran T40 4%

Amino Acids 2%

CaCO3 80 mg

Xanthane 0.3%

In addition to that some samples contain adjuvant:

| | |
|---|---|
| Sample 1 | No adjuvant |
| Sample 2 | LT 25 μg |
| Sample 3 | Laureth-9 0.5% |
| Sample 4 | MPL 5 μg |
| Sample 5 | Laureth-9 0.5%, MPL 5 μg |

Placebos have also been prepared containing everything exept the flu whole virus

Gels and western blotting show that the HA keeps its integrity after lyophilisation. SRD assay to quantify the HA has been performed and gives the expected HA values.

Groups of 8 mice (Femele Balb/c 6 weeks old) were primed intranasally with 5 μg/HA of whole inactivated antigen (H1N1 A/Beijing/262/95) and were orally immunized (except group 1: intramuscular injection) 28 days later with the following formulations containing 3 μg HA of the same whole inactivated antigen. Sera and feces were collected before the first dose, 14, 42 and 56 days after. All sera were tested for their specific anti-Beijing IgG activity by ELISA and for their hemagglutination inhibition capacity (HI assay). The detection of specific anti-Beijing IgA was conducted on the feces using two separate ELISAs (total IgA quantification in μg/ml and specific anti-Beijing end-point titers). The final results were expressed as a ratio between specific IgA and total IgA.

Results

Adjuvanted Lyoc formulations containing either LT or 3D-MPL are able to elicit a specific humoral immune response specific for influenza, with HI titres of approximately 50. All lyoc formulations induced a significant immune response after each boosting, the magnitude of the observed peak immune responses after each subsequent boosting dose was greater than the peak observed after the previous boosting dose.

### EXAMPLE 8

Lyophilised Formulations.

Preferred formulations are the result of a compromise between different physico-chemical properties. In preferred formulations:

The lyophilised cake is strong enough to support manufacturing handling and manipulations during administration.

It should not be affected by the humidity of the hand, when administered.

It must be light enough in order to dissolve instantaneously when placed in the mouth.

Specific formulations may vary depending upon the presence of an antacid. By way of example:

A Formulations without Antacid.

In this cake, the lyophilised cake generally dissolves very quickly when placed in the mouth. So it is preferred that the lyophilised cake is strong enough to be manipulated.

Suitable formulations include

| Batch | Sucrose | Dextran | Sorbitol | Am-acids | volume | weight | dissolution |
|---|---|---|---|---|---|---|---|
| 00L15L/01 | 2% | 4%; 10000 | 3% | 2% | 0.4 ml | 44 mg | <5 sec |
| 00L15L/02 | 2% | 2%; 10000 | 3% | 2% | 0.4 ml | 36 mg | <5 sec |
| 00L15L/03 | 2% | 4%; 40000 | 3% | 2% | 0.4 ml | 44 mg | <5 sec |
| 00L15L/04 | 2% | 2%; 40000 | 3% | 2% | 0.4 ml | 36 mg | <5 sec |
| 00L15L/05 | 2% | 3%; 70000 | 3% | 2% | 0.4 ml | 40 mg | <5 sec |
| 00L15L/06 | 2% | 2%; 70000 | 3% | 2% | 0.4 ml | 36 mg | <5 sec |
| 00L15L/07 | 2% | 1%; 70000 | 3% | 2% | 0.4 ml | 32 mg | <5 sec |
| 00L15L/08 | 2% | 0.5% 70000 | 3% | 2% | 0.4 ml | 30 mg | <5 sec |
| 01A19/01 | 2% | 4% 10000 | 3% | 2% | 0.4 ml | 44 mg | <5 sec |
| 01A19/02 | 2% | 6% 10000 | 3% | 2% | 0.4 ml | 52 mg | <5 sec |
| 01A19/03 | 2% | 8% 10000 | 3% | 2% | 0.4 ml | 60 mg | <5 sec |
| 01A19/04 | 2% | 10% 10000 | 3% | 2% | 0.4 ml | 68 mg | <10 sec |
| 01A19/05 | 1% | 8% 10000 | 1% | 2% | 0.4 ml | 48 mg | <5 sec |
| 01A19/06 | 1% | 10% 10000 | 1% | 2% | 0.4 ml | 56 mg | <10 sec |
| 01A19/07 | 2% | 4% 40000 | 3% | 2% | 0.4 ml | 44 mg | <5 sec |
| 01A19/08 | 2% | 6% 40000 | 3% | 2% | 0.4 ml | 52 mg | <5 sec |
| 01A19/09 | 2% | 8% 40000 | 3% | 2% | 0.4 ml | 60 mg | <10 sec |
| 01A19/10 | 1% | 6% 40000 | 1% | 2% | 0.4 ml | 40 mg | <10 sec |
| 01A19/11 | 1% | 8% 40000 | 1% | 2% | 0.4 ml | 48 mg | <10 sec |
| 01A19/12 | 1% | 3% 40000 | 1% | 2% | 0.4 ml | 28 mg | <5 sec |
| 01B09/1 | 3% | 3% 40000 | 2% | 3% | 0.4 ml | 44 mg | <10 sec |
| 01B09/2 | 2% | 3% 40000 | 2% | 4% | 0.4 ml | 44 mg | <5 sec |
| 01B09/3 | 2% | 3% 40000 | 3% | 3% | 0.4 ml | 44 mg | <5 sec |
| 01B09/4 | 3% | 3% 40000 | 3% | 2% | 0.4 ml | 44 mg | <5 sec |
| 01B09/5 | 2.5% | 3% 40000 | 3% | 2.5% | 0.4 ml | 44 mg | <5 sec |
| 01B09/6 | 2% | 4% 40000 | 3% | 2% | 0.4 ml | 44 mg | <5 sec |
| 01B09/7 | 2.0% | 5% 40000 | 2.0% | 2.0% | 0.4 ml | 44 mg | <5 sec |
| 01B09/8 | 3.0% | 5% 40000 | 1.0% | 2.0% | 0.4 ml | 44 mg | <5 sec |
| 01B09/9 | 2.0% | 5% 40000 | 1.0% | 3.0% | 0.4 ml | 44 mg | <5 sec |
| 01B09/10 | 2% | 6% 40000 | 2% | 1% | 0.4 ml | 44 mg | <5 sec |
| 01B09/11 | 1% | 6% 40000 | 2% | 2% | 0.4 ml | 44 mg | <10 sec |
| 01B09/12 | 2% | 6% 40000 | 1% | 2% | 0.4 ml | 44 mg | <10 sec |
| 01B16/1 | 4% | 4% 40000 | 2.66 | 4% | 0.3 ml | 44 mg | |
| 01B16/2 | 2.66% | 4% 40000 | 2.66 | 5.33% | 0.3 ml | 44 mg | |
| 01B16/3 | 2.66% | 4% 40000 | 4% | 4% | 0.3 ml | 44 mg | |
| 01B16/4 | 4% | 4% 40000 | 4% | 2.66% | 0.3 ml | 44 mg | |
| 01B16/5 | 3.33% | 4% 40000 | 4% | 3.33% | 0.3 ml | 44 mg | |
| 01B16/6 | 2.66% | 5.33% 40000 | 4% | 2.66% | 0.3 ml | 44 mg | |
| 01B16/7 | 2.66% | 6.66% 40000 | 2.66% | 2.66% | 0.3 ml | 44 mg | |
| 01B16/8 | 4.0% | 6.66% 40000 | 1.33% | 2.66% | 0.3 ml | 44 mg | |
| 01B16/9 | 2.66% | 6.66% 40000 | 1.33% | 4.0% | 0.3 ml | 44 mg | |
| 01B16/10 | 2.66% | 8% 40000 | 2.66% | 1.33% | 0.3 ml | 44 mg | |
| 01B16/11 | 1.33% | 8% 40000 | 2.66% | 2.66% | 0.3 ml | 44 mg | |
| 01B16/12 | 2.66% | 8% 40000 | 1.33% | 2.66% | 0.3 ml | 44 mg | |

Where necessary, to support manufacturing or administration handling, increasing cake solidity can be achieved by addition of polymeric substance like Xanthane, Kelgum 100, Kelgum GFS, or Pectine.

| Batch | Sucrose | Dextran | Sorbitol | Am-acids | Xanthane | Kelgum | volume | weight | dissolution |
|---|---|---|---|---|---|---|---|---|---|
| 01D06/01 | 2% | T40:4% | 3% | 2% | 10 mg | | | | |
| 01D06/02 | 2% | T40:4% | 3% | 2% | 20 mg | | | | |
| 00K24/02 | 2% | 4%; 5000 | 3% | 2% | 0.33% | | 0.4 ml | 45.32 mg | <10 sec |
| 00K24/04 | 2% | 4%; 10000 | 3% | 2% | 0.33% | | 0.4 ml | 45.32 mg | <10 sec |
| 00K24/06 | 2% | 4%; 40000 | 3% | 2% | 0.33% | | 0.4 ml | 45.32 mg | <20 sec |
| 00K24/08 | 2% | 4%; 70000 | 3% | 2% | 0.33% | | 0.4 ml | 45.32 mg | <10 sec |
| 00K2410 | 2% | 4%; 5000 | 3% | 2% | | 0.167 | 0.4 ml | 44.67 mg | <15 sec |
| 00K24/12 | 2% | 4%; 10000 | 3% | 2% | | 0.167 | 0.4 ml | 44.67 mg | <10 sec |
| 00K24/14 | 2% | 4%; 40000 | 3% | 2% | | 0.167 | 0.4 ml | 44.67 mg | <10 sec |
| 00K24/16 | 2% | 4%; 70000 | 3% | 2% | | 0.167 | 0.4 ml | 44.67 mg | <10 sec |

| Batch | Sucrose | Dextran | Sorbitol | Am-acids | | volume | weight | dissolution |
|---|---|---|---|---|---|---|---|---|
| 01C16/02 | 2.38% | 4.76% | 3.57% | 2.38% | pectine 0.5% | 0.4 ml | 54.76 mg | <5 sec |
| 01C16/03 | 2% | 4% | 3% | 2% | pectine 0.5% | 0.5 ml | 57.50 mg | <5 sec |

Formulation 01C16/03 is particularly preferred.

B Formulations with Antacid

When using an antacid like CaCO₃, it is preferred to maintain homogeneity of the suspension during the filling steps.

This can be achieved by:

increasing the viscosity of the medium (using for example: Xanthane, Kelgum or Pectine)

Increasing the thickness of the suspension (by using for example: Starch)

Creating gel in the medium (by cross-linking pectine with calcium ion).

Suitable formulations include

| Batch | Sucrose | Dextran | Sorbitol | Am-acids | CaCO₃ | Starch |
|---|---|---|---|---|---|---|
| 00J11/01 | 2% | 4%; 10000 | 3% | 2% | 80 mg | 1.50% |
| 00J11/02 | 1% | 2%; 10000 | 1.50% | 1% | 80 mg | 1.50% |
| 00K17/01 | 2% | 4%; 5000 | 3% | 2% | 80 mg | 1.50% |
| 00K17/02 | 2% | 4%; 10000 | 3% | 2% | 80 mg | 1.50% |
| 00K17/03 | 2% | 4%; 40000 | 3% | 2% | 80 mg | 1.50% |
| 00K17/04 | 2% | 4%; 70000 | 3% | 2% | 80 mg | 1.50% |
| 00K17/05 | 1% | 2%; 70000 | 1.50% | 1% | 80 mg | 1.50% |

| Batch | Sucrose | Dextran | Sorbitol | Am-acids | CaCO₃ | Xanthane | Kelgum | Starch |
|---|---|---|---|---|---|---|---|---|
| 00K24/01 | 2% | 4%; 5000 | 3% | 2% | 80 mg | 0.33% | | |
| 00K24/03 | 2% | 4%; 10000 | 3% | 2% | 80 mg | 0.33% | | |
| 00K24/05 | 2% | 4%; 40000 | 3% | 2% | 80 mg | 0.33% | | |
| 00K24/07 | 2% | 4%; 70000 | 3% | 2% | 80 mg | 0.33% | | |
| 00K24/09 | 2% | 4%; 5000 | 3% | 2% | 80 mg | | 0.167 | |
| 00K24/11 | 2% | 4%; 10000 | 3% | 2% | 80 mg | | 0.167 | |
| 00K24/13 | 2% | 4%; 40000 | 3% | 2% | 80 mg | | 0.167 | |
| 00K24/15 | 2% | 4%; 70000 | 3% | 2% | 80 mg | | 0.167 | |
| 00L01/01 | 2% | 4%; 40000 | 3% | 2% | 80 mg | 0.33% | | |
| 00L01/02 | 2% | 3%; 40000 | 3% | 2% | 80 mg | 0.33% | | |
| 00L01/03 | 2% | 2%; 40000 | 3% | 2% | 80 mg | 0.33% | | |
| 00L01/04 | 2% | 1%; 40000 | 3% | 2% | 80 mg | 0.33% | | |
| 00L01/05 | 2% | 4%; 40000 | 3% | 2% | 80 mg | | 0.17% | |
| 00L01/06 | 2% | 3%; 40000 | 3% | 2% | 80 mg | | 0.17% | |
| 00L01/07 | 2% | 2%; 40000 | 3% | 2% | 80 mg | | 0.17% | |
| 00L01/08 | 2% | 1%; 40000 | 3% | 2% | 80 mg | | 0.17% | |
| 00L01/09 | 2% | 4%; 40000 | 3% | 2% | 80 mg | | | 1.50% |
| 00L01/10 | 2% | 3%; 40000 | 3% | 2% | 80 mg | | | 1.50% |
| 00L01/11 | 2% | 2%; 40000 | 3% | 2% | 80 mg | | | 1.50% |
| 00L01/12 | 2% | 1%; 40000 | 3% | 2% | 80 mg | | | 1.50% |
| 00L01/13 | 2% | 4%; 70000 | 3% | 2% | 80 mg | 0.33% | | |
| 00L01/14 | 2% | 3%; 70000 | 3% | 2% | 80 mg | 0.33% | | |
| 00L01/15 | 2% | 2%; 70000 | 3% | 2% | 80 mg | 0.33% | | |
| 00L01/16 | 2% | 1%; 70000 | 3% | 2% | 80 mg | 0.33% | | |
| 00L01/17 | 2% | 4%; 70000 | 3% | 2% | 80 mg | | 0.17% | |
| 00L01/18 | 2% | 3%; 70000 | 3% | 2% | 80 mg | | 0.17% | |
| 00L01/19 | 2% | 2%; 70000 | 3% | 2% | 80 mg | | 0.17% | |
| 00L01/20 | 2% | 1%; 70000 | 3% | 2% | 80 mg | | 0.17% | |
| 00L01/21 | 2% | 4%; 70000 | 3% | 2% | 80 mg | | | 1.50% |
| 00L01/2 | 2% | 3%; 70000 | 3% | 2% | 80 mg | | | 1.50% |
| 00L01/23 | 2% | 2%; 70000 | 3% | 2% | 80 mg | | | 1.50% |
| 00L01/24 | 2% | 1%; 70000 | 3% | 2% | 80 mg | | | 1.50% |
| 00L08/01 | 2% | 2%; 70000 | 3% | 2% | 80 mg | | | 1.20% |
| 00L08/02 | 2% | 2%; 70000 | 3% | 2% | 80 mg | | | 1.20% |
| 00L08/03 | 2% | 2%; 70000 | 3% | 2% | 80 mg | 0.20% | | |
| 00L08/04 | 2% | 2%; 70000 | 3% | 2% | 80 mg | 0.20% | | |
| 00L08/05 | 2% | 2%; 70000 | 3% | 2% | 80 mg | | 0.13% | |
| 00L08/06 | 2% | 2%; 70000 | 3% | 2% | 80 mg | | 0.13% | |
| 00L08/07 | 2% | 3%; 70000 | 3% | 2% | 80 mg | | | 1.20% |
| 00L08/08 | 2% | 3%; 70000 | 3% | 2% | 80 mg | | | 1.20% |
| 00L08/09 | 2% | 3%; 70000 | 3% | 2% | 80 mg | 0.20% | | |
| 00L08/10 | 2% | 3%; 70000 | 3% | 2% | 80 mg | 0.20% | | |
| 00L08/11 | 2% | 3%; 70000 | 3% | 2% | 80 mg | | 0.13% | |
| 00L08/12 | 2% | 3%; 70000 | 3% | 2% | 80 mg | | 0.13% | |

| Batch | Sucrose | Dextran | Sorbitol | Am-acids | | CaCO₃ | |
|---|---|---|---|---|---|---|---|
| 01D20/04 | 2% | T40:4% | 3% | 2% | Xanthane 0.012% | 80 mg | |
| 01D20/05 | 2% | T40:4% | 3% | 2% | Kelgum 100: 0.012% | 80 mg | |
| 01D20/06 | 2% | T40:4% | 3% | 2% | Kelgum GFS 0.012% | 80 mg | |
| 01D20/07 | 2% | T40:4% | 3% | 2% | Xanthane 0.008% | 80 mg | |
| 01D20/08 | 2% | T40:4% | 3% | 2% | Kelgum 100: 0.008% | 80 mg | |
| 01C16/01 | 2% | 4% | 3% | 2% | pectine 0.5% | 80 mg | |
| 01C16/04 | 2% | 4% | 3% | 2% | inuline 5% | 80 mg | |
| 01C16/07 | 2% | 4% | 3% | 2% | inuline 10% | 80 mg | |
| 01C23/01 | 2% | T10:4% | 3% | 2% | pectine 0.5% | 80 mg | |
| 01C23/02 | no | T10:4% | 3% | 2% | pectine 0.5% | 80 mg | |
| 01C23/03 | no | T40:4% | 3% | 2% | pectine 0.5% | 80 mg | |
| 01C23/04 | 2% | T40:4% | 3% | 2% | pectine 0.5% | 80 mg | |
| 01C23/05 | 2% | T40:4% | 3% | 2% | pectine 0.5% | 80 mg | Tri—Ca- |
| | | | | | | | dicitrate |
| 01C23/06 | 2% | T40:4% | 3% | 2% | pectine 0.5% | 80 mg | CaCl₂ |
| 01C30/01 | 2% | T40:4% | 3% | 2% | pectine 0.5% | 80 mg | |
| 01C30/02 | 2% | T40:4% | 3% | 2% | pectine 0.25% | 80 mg | |
| 01C30/03 | 2% | T40:4% | 3% | 2% | pectine 0.1% | 80 mg | |

CaCO₃ Merck product n° 102069 (particles size 3 μm) gives better results that Merck product n° 112120 (particles size: 30 μm) and particles of substantially 3 μm are thus preferred.

| | | | | | CaCO₃ Merck n° |
|---|---|---|---|---|---|
| Batch | Sucrose | Dextran | Sorbitol | Am-acids | 102069 (3 μm) |
| 01F06/01 | 4% | T10: 8% | 6% | 4% | 80 mg |
| 01F06/02 | 2% | T10: 4% | 3% | 2% | 80 mg |
| 01F06/03 | 4% | T40: 8% | 6% | 4% | 80 mg |
| 01F06/04 | 2% | T40: 10% | 3% | 2% | 80 mg |

As can be seen from the aove tables, preferred formulations comprise sucrose, dextran, sorbitol and amino acids, suitably in ranges given above.

1. An oral solid dose vaccine composition, comprising an antigen, and suitable excipients, wherein the solid dose vaccine is in the form of a quick dissolving cake which dissolves in less than 10 seconds, characterised in that the composition further comprises a water insoluble antacid that also acts as an adjuvant, wherein the antigen is adsorbed onto the surface of the antacid.

2. An oral solid dose vaccine composition as claimed in claim 1, wherein the water insoluble antacid is calcium carbonate.

3. An oral solid dose vaccine composition as claimed in claim 2, wherein the calcium carbonate is present in a particle size of substantially 3 μm.

4. An oral solid dose vaccine composition as claimed in any one of claims 1 to 3, wherein the vaccine comprises more than one antigen.

5. An oral solid dose vaccine composition as claimed in claim 3 wherein the more than one antigen are from more than one pathogen.

6. An oral solid dose vaccine composition as claimed in any one of claims 1 to 5, further comprising dextran.

7. An oral solid dose vaccine composition as claimed in and one of claims 1 to 6, further comprising a live attenuated bacterial or viral vaccine.

8. An oral solid dose vaccine composition as claimed in any one of claims 1 to 7, wherein the quick dissolving cake is formed by sublimation of a liquid vaccine composition.

9. An oral solid dose vaccine composition as claimed in any one of claims 1 to 8 wherein the vaccine composition comprises an additional antacid.

10. An oral solid dose vaccine composition as claimed in claim 9, wherein the additional antacid is selected from aluminium hydroxide or magnesium hydroxide.

11. An oral solid dose vaccine composition as claimed in claim 6, wherein the antacid is a combination of aluminium hydroxide and magnesium hydroxide.

12. An oral solid dose vaccine composition as claimed in any one of claims 1 to 11, wherein the vaccine composition comprises a binding agent.

13. An oral solid dose vaccine composition as claimed in claim 12, wherein the binding agent is dextran.

14. An oral solid dose vaccine composition as claimed in any one of claims 1 to 13, wherein the vaccine formulation comprises a stabilising glass forming polyol.

15. An oral solid dose vaccine composition as claimed in claim 14 wherein the glass forming polyol is selected from trehalose, sucrose, lactose, fructose, galactose, mannose, maltulose, iso-maltulose and lactulose, maltose, or dextrose and sugar alcohols of the aforementioned such as mannitol, lactitol and maltitol.

16. An oral solid dose vaccine composition as claimed in any one of claims 1 to 15, wherein the vaccine composition comprises a pseudoplastic excipient or thixotropic agent.

17. An oral solid dose vaccine composition as claimed in claim 16, wherein the pseudoplastic excipient is xanthane gum.

18. An oral solid dose vaccine composition as claimed in claim 1, comprising xanthane gum, dextran and calcium carbonate.

19 An oral solid dose vaccine composition as claimed in claim 1, comprising xanthane gum, dextran and aluminium hydroxide.

20. An oral solid dose vaccine composition as claimed in claims 18 or 19, additionally comprising sorbitol.

21. An oral solid dose vaccine composition as claimed in any one of claims 1 to 20, wherein the antigen or antigen composition is derived from the group comprising: Human Immunodeficiency Virus, Varicella Zoster virus, Herpes Simplex Virus type 1, Herpes Simplex virus type 2, Human cytomegalovirus, Dengue virus, Hepatitis A, B, C or E, Respiratory Syncytial virus, human papilloma virus, Influenza virus, Hib, Meningitis virus, Salmonella, Neisseria, Borrelia, Chlamydia, Bordetella, Plasmodium or Toxoplasma, stanworth decapeptide; or Tumor associated antigens (TMA), MAGE, BAGE, GAGE, MUC-1, Her-2 neu, LnRH, CEA, PSA, KSA, or PRAME.

22. An oral solid dose vaccine composition as claimed in any one of claims 1 to 21, wherein the vaccine composition additionally comprises an adjuvant.

23. An oral solid dose vaccine composition as claimed in claim 22, wherein the adjuvant is selected from: LT, CT, 3D-MPL, CpG, QS21.

24. An oral solid dose vaccine composition, comprising an antigen, calcium carbonate and suitable excipients, wherein the solid dose vaccine is in the form of a quick dissolving cake and wherein the calcium carbonate is present in a particle size of substantially 3 μm.
